SEQUENCE LISTING (16 sequences)

SEQ ID NO: 1 - DNA, 357 bp, Artificial
  Synthetic construct  coding sequence of heavy chain ScFV (VH)
gaggtcaagctgcaggagtcaggaactgaagtggtaaagcctggggcttcagtgaagttgtcctgcaaggcttctggctacatcttcacaagttatgatatagactgggtgaggcagacgcctgaacagggacttgagtggattggatggatttttcctggagaggggagtactgaatacaatgagaagttcaagggcagggccacactgagtgtagacaagtcctccagcacagcctatatggagctcactaggctgacatctgaggactctgctgtctatttctgtgctagaggggactactataggcgctactttgacttgtggggccaagggaccacggtcaccgtctcctca

SEQ ID NO: 2 - DNA, 321 bp, Artificial
  Synthetic construct  coding sequence of light chain ScFv (VL)
gaaaatgtgctcacccagtctccagcaatcatgtctgcatctccaggggagagggtcaccatgacctgcagtgccagctcaagtatacgttacatatattggtaccaacagaagcctggatcctcccccagactcctgatttatgacacatccaacgtggctcctggagtcccttttcgcttcagtggcagtgggtctgggacctcttattctctcacaatcaaccgaatggaggctgaggatgctgccacttattactgccaggagtggagtggttatccgtacacgttcggaggggggaccaagctggagctgaaagcg

SEQ ID NO: 3 - DNA, 768 bp, Artificial
  Synthetic construct  coding sequence of TNOX-specific ScFv (single chain antibody)
gaggtcaagctgcaggagtcaggaactgaagtggtaaagcctggggcttcagtgaagttgtcctgcaaggcttctggctacatcttcacaagttatgatatagactgggtgaggcagacgcctgaacagggacttgagtggattggatggatttttcctggagaggggagtactgaatacaatgagaagttcaagggcagggccacactgagtgtagacaagtcctccagcacagcctatatggagctcactaggctgacatctgaggactctgctgtctatttctgtgctagaggggactactataggcgctactttgacttgtggggccaagggaccacggtcaccgtctcctcaggaggcggtggatcgggcggtggcggctcgggtggcggcggctctgaaaatgtgctcacccagtctccagcaatcatgtctgcatctccaggggagagggtcaccatgacctgcagtgccagctcaagtatacgttacatatattggtaccaacagaagcctggatcctcccccagactcctgatttatgacacatccaacgtggctcctggagtcccttttcgcttcagtggcagtgggtctgggacctcttattctctcacaatcaaccgaatggaggctgaggatgctgccacttattactgccaggagtggagtggttatccgtacacgttcggaggggggaccaagctggagctgaaagcgaaagaaaccgctgctgctaaattcgaacgccagcacatggacagc

SEQ ID NO: 4 - protein, 256 aa, Artificial
  Synthetic construct  amino acid sequence of ScFv (single chain antibody) specific for tNOX, ECTO-NOX
EVKLQESGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSSGGGGSGGGGSGGGGSENVLTQSPAIMSASPGERVTMTCSASSSIRYIYWYQQKPGSSPRLLIYDTSNVAPGVPFRFSGSGSGTSYSLTINRMEAEDAATYYCQEWSGYPYTFGGGTKLELKAKETAAAKFERQHMDS

SEQ ID NO: 5 - protein, 15 aa, Artificial
  Synthetic construct  linker sequence for joining heavy and light chains in single chain antibody specific for tNOX (ECTO-NOX)
GGGGSGGGGSGGGGS

SEQ ID NO: 6 - DNA, 45 bp, Artificial
  Synthetic construct  oligonucleotide sequence encoding linker
ggaggcggtggatcgggcggtggcggctcgggtggcggcggctct

SEQ ID NO: 7 - DNA, 45 bp, Artificial
  Synthetic construct  oligonucleotide encoding S peptide
aaagaaaccgctgctgctaaattcgaacgccagcacatggacagc

SEQ ID NO: 8 - protein, 15 aa, Artificial
  Synthetic construct  amino acid sequence of S peptide
KETAAAKFERQHMDS

SEQ ID NO: 9 - DNA, 37 bp, Artificial
  Synthetic construct  oligonucleotide useful as a primer
ggcccagccggccgaggtcaagctgcaggagtcagga

SEQ ID NO: 10 - DNA, 33 bp, Artificial
  Synthetic construct  oligonucleotide useful as a primer
ctcggaacctgaggagacggtgaccgtggtccc

SEQ ID NO: 11 - DNA, 36 bp, Artificial
  Synthetic construct  oligonucleotide useful as a primer
tccaaagtcgacgaaaatgtgctcacccagtctcca

SEQ ID NO: 12 - DNA, 33 bp, Artificial
  Synthetic construct  oligonucleotide useful as a primer
agcggccgctttcagctccagcttggtcccccc

SEQ ID NO: 13 - DNA, 21 bp, Artificial
  Synthetic construct  oligonucleotide useful as a  primer
gtcaagctgcaggagtcagga

SEQ ID NO: 14 - DNA, 66 bp, Artificial
  Synthetic construct  oligonucleotide useful as a primer
agagccgccgccacccgagccgccaccgcccgatccaccgcctcctgaggagacggtgaccgtggt

SEQ ID NO: 15 - DNA, 66 bp, Artificial
  Synthetic construct  oligonucleotide useful as a primer
ggaggcggtggatcgggcggtggcggctcgggtggcggcggctctgaaaatgtgctcacccagtct

SEQ ID NO: 16 - DNA, 78 bp, Artificial
  Synthetic construct  oligonucleotide useful as a primer
agtcaggctagcttagctgtccatgtgctggcgttcgaatttagcagcagcggtttctttcgctttcagctccagctt

(19) United States
(12) Patent Application Publication
(10) Pub. No.: US 2009/0042209 A1
(43) Pub. Date: Feb. 12, 2009

(54) Neoplasia-Specific tNOX Isoforms and Methods

(75) Inventor: Brandon Hostetler, Walkerton, IN (US)

Correspondence Address: GREENLEE WINNER AND SULLIVAN P C, 4875 PEARL EAST CIRCLE, SUITE 200, BOULDER, CO, 80301, US

(73) Assignee: NOX TECHNOLOGIES, INC., WEST LAFAYETTE, IN (US)

(21) Appl. No.: 11/848,942

(22) Filed: Aug. 31, 2007

Related U.S. Application Data
(60) Provisional application No. 60/824,398, filed on Sep. 1, 2006.
(60) Provisional application No. 60/824,333, filed on Sep. 1, 2006.

(51) Int. Cl. G01N 33/574

(52) U.S. Cl. 435/7.1

(57) ABSTRACT

All neoplastic cells express one or more members of a unique family of cell surface ubiquinone (NADH) oxidase proteins with protein disulfide-thiol interchange activity (ECTO-NOX proteins) that are characteristically inhibited by quinone site inhibitors with anti-cancer activity. Cancers of different cellular or tissue origins express different tNOX cancer isoforms or combinations of isoforms and shed these proteins into the circulation. Herein are disclosed methods both for cancer detection and diagnosis of particular origin, based on the patterns and molecular weights of the isoforms which allow the identification of the cell type and or tissue of origin of the neoplasm. Relative tNOX amounts are proportional to tumor burden and provide a reliable measure of response to therapy and disease progression.

### CROSS-REFERENCE TO RELATED APPLICATIONS

This application claims benefit of U.S. Provisional Application 60/824,398 and U.S. Provisional Application 60/824,333, both filed Sep. 1, 2006, and both incorporated by reference herein to the extent there is no inconsistency with the present disclosure.

### BACKGROUND OF THE INVENTION

The field of this invention is the area of protein biochemistry, in particular, as related to the diagnosis of neoplastic and diseased cells, as specifically related to particular isoforms of a cell surface marker characteristic of neoplasia in general and with specific patterns of protein expression indicative of specific types of cancer. Detection of the particular isoforms is by use of specific antibodies.

There is a unique, growth-related family of cell surface hydroquinone or NADH oxidases with protein disulfide-thiol interchange activity referred to as ECTO-NOX proteins (for cell surface NADH oxidases) (1,2). One member of the ECTO-NOX family, designated tNOX (for tumor associated) is specific to the surfaces of cancer cells and the sera of cancer patients (3, 4). The presence of the tNOX protein has been demonstrated for several human tumor tissues (mammary carcinoma, prostate cancer, neuroblastoma, colon carcinoma and melanoma) (5), and serum analysis suggest a much broader association with human cancer (6, 7).

NOX proteins are ectoproteins anchored in the outer leaflet of the plasma membrane (8). As is characteristic of other examples of ectoproteins (sialyl and galactosyl transferase, dipeptidylamino peptidase IV, etc.), the NOX proteins are shed. They appear in soluble form in conditioned media of cultured cells (5) and in patient sera (6, 7). The serum form of tNOX from cancer patients exhibits the same degree of drug responsiveness as does the membrane-associated form. Drug-responsive tNOX activities are seen in sera of a variety of human cancer patients, including patients with leukemia, lymphomas or solid tumors (prostate, breast, colon, lung, pancreas, ovarian, liver) (6, 7). An extreme stability and protease resistance of the tNOX protein (9) may help explain its ability to accumulate in sera of cancer patients to readily detectable levels. In contrast, no drug-responsive NOX activities have been found in the sera of healthy volunteers (6, 7) or in the sera of patients with disorders other than neoplasia.

While the basis for the cancer specificity of cell surface tNOX has not been determined, the concept is strongly supported by several lines of evidence. A drug responsive tNOX activity has been rigorously determined to be absent from plasma membranes of non-transformed human and animal cells and tissues (3). The tNOX proteins lack a transmembrane binding domain (10) and are released from the cell surface by brief treatment at low pH (9). A drug-responsive tNOX activity has not been detected in sera from healthy volunteers or patients with diseases other than cancer (6, 7). Several tNOX antisera have identified the immunoreactive band at 34kDa (the processed molecular weight of one of the cell surface forms of tNOX) with Western blot analysis or immunoprecipitation when using transformed cells and tissues or sera of cancer patients as antigen source (5,10,11). The immunoreactive band at 34kDa is absent with western blot analysis or immunoprecipitation when using transformed cells and tissues or sera from healthy volunteers or patents with disorders other than cancer (5,10,11). These antisera include a monoclonal antibody (5), single-chain variable region fragment (scFv) which reacts with the cell surface NADH oxidase from normal and neoplastic cells, polyclonal antisera made in response to expressed tNOX (11) and polyclonal peptide antisera to the conserved adenine nucleotide binding region of tNOX (11).

tNOX cDNA has been cloned (GenBank Accession No. AF207881; 11; U.S. Patent Publication 2003-0207340 A1). The derived molecular weight from the open reading frame was 70.1 kDa. Functional motifs include a quinone binding site, an adenine nucleotide binding site, and a CXXXXC cysteine pair as a potential protein disulfide-thiol interchange site based on site-directed mutagenesis (11). Based on available genomic information (12) the tNOX gene is located on chromosome X, and it is comprised of multiple exons (thirteen). It is known that there are a number of splice variant mRNAs and proteins expressed.

The hybridoma cell line which produces the tumor NADH oxidase-specific monoclonal antibody MAB 12.1 was deposited with the American Type Culture Collection, Manassas, Va., 20108 on Apr. 4, 2002, under the terms of the Budapest Treaty. This deposit is identified by Accession No. ATCC PTA-4206. The deposit will be maintained with restriction in the ATCC depository for a period of 30 years from the deposit date, or 5 years after the most recent request, or for the effective life of the patent, which ever is longer, and will be replaced if the deposit becomes non-viable during that period. This monoclonal antibody is described in U.S. Pat. No. 7,053,188, issued May 30, 2006, which is incorporated by reference herein.

Because cancer poses a significant threat to human health and because cancer results in significant economic costs, there is a long-felt need in the art for an effective, economical and technically simple system in which to assay for the presence of cancer.

### BRIEF SUMMARY OF THE INVENTION

The present invention provides a method for the analysis of a biological sample for the presence of particular isoforms of the pan-cancer antigen known as tNOX (for tumor-specific NADH oxidase). The present method entails 2-dimensional gel electrophoresis and immunoblotting using an antibody specific for the pan-cancer tNOX antigen and the various isoforms which characterize particular types of cancers. As specifically exemplified, about 4-6 mg protein are loaded for analysis.

The present invention provides a method for the detection of particular cancer-specific serum or plasma tNOX isoforms as indicators of the presence of cancer and of the cell type or tissue of cancer origin (breast, ovarian prostate, etc). tNOX isoforms are identified on the basis of their molecular mass and isoelectric point with detection using a tNOX-specific monoclonal antibody (MAB) (U.S. Pat. No. US 7,053,188), using single chain variable region (ScFv) fragment which recognizes all cell surface NOX proteins (both age-related, normal cell and neoplasia specific NADH oxidase) or using polyclonal sera raised against tNOX. The ECTO-NOX proteins are first enriched and concentrated from a biological sample, desirably a serum sample, by binding to nickel-agarose and then eluting. After release of the proteins from the nickel-agarose by vortexing, the proteins are separated in the first dimension by isoelectric focusing and in the second dimension by polyacrylamide gel electrophoresis. As specifically exemplified herein, the isoelectric focusing step is over a pH range from 3 to 10, and size separation is over a 10% polyacrylamide gel. Most of the cancer-specific tNOX isoforms exhibit isoelectric points in a very narrow range between pH 4.4 and 5.4 but differ in molecular weight from 34 to 136 kDa. In the 2D gel system specifically exemplified, the cancer-specific isoforms are located in Quadrants I (relatively high molecular weight material) and IV (lower molecular weight material, notably the 34 and 43 kDa isoforms). IgG heavy chains (Quadrant II and IgG light chains (Quadrant III) cross react with the ScFv antibody and serve as loading controls. The absence of all tNOX isoforms indicates the absence of cancer; the presence of a tNOX isoform indicates the presence of cancer. The particular molecular weight present in a serum sample or a particular combination of isoforms provides an indication of the cell type or tissue of origin of the cancer. The method not only determines cancer presence, but also the method of the present invention provides diagnostic information concerning the tissue of origin. At present there are no other pan cancer (all forms of human cancer) tests with these particular capabilities.

The present invention provides a method for determining neoplasia in a mammal, including a human, said method comprising the steps of detecting cancer presence, in a biological sample. The present invention further provides additional information for assessment of neoplasia, including a measure of tumor burden, for example in serum, plasma, urine, saliva or in biopsy material.

Also within the scope of the present invention are particular isoforms of tNOX associated with specific (primary) cancers: tNOX proteins with apparent molecular weights of about 64, 66 and/or 68 kDa are associated with breast cancer; two tNOX proteins of about 40.5 and 52 kDa are associated with small cell lung cancer; tNOX proteins of about 40.5, 52 and 80 kDa characterize ovarian cancer; two tNOX isoforms of about 75 kDa designated 75α and 75β are associated with prostate cancer; a tNOX protein of about 94 kDa is associated with cervical cancer; tNOX proteins of about 43 and 52 kDa are characteristic of colon cancer; and a tNOX isoform of about 54 kDa is associated with non small cell lung cancer. Where a patient is suspected of having cancer, a biological sample, advantageously a serum sample can be prepared, and the 2D gel electrophoresis/immunological analysis of the present invention can be undertaken. Positive results are indicative of the presence of cancer, and the detection of characteristic proteins allow a presumption as to the primary incidence of cancer in that patient according to the association of particular protein(s) with particular cancer origins, as set forth above.

The methods of the present invention can also be applied to evaluate response to therapy, with decreasing amounts of NOX isoform(s) reflecting successful treatment, as well as early detection of recurrent disease (reflected increased or reappearance of tNOX-specific isoforms.

### BRIEF DESCRIPTION OF THE DRAWINGS

FIG. 1 provides the results of analytical 2-D gel electrophoresis of ECTO-NOX enriched serum proteins from a preparation of sera pooled from cancer patients (breast, ovarian, lung and colon) concentrated by nickel agarose precipitation, followed by immunoblots analysis. Separation in the first dimension was by isoelectric focusing and in the second dimension by SDS-PAGE (using ampholytes over the pH range of 3 to 10 and 10% polyacrylamide gel). For interpretative purposes, the gel is divided into quadrants I-IV with unreactive albumin (albumin) at the center. Detection was with recombinant anti-tNOX single chain variable region (scFv) antibody (MAB 12.1 scFv) carrying an S-tag followed by alkaline phosphatase-liked anti S (Novagen cat. # 69598-3 or equivalent product) with Western Blue NBT substrate (Promega, Madison, Wis; Cat. No. S3841 or equivalent product). Reactive proteins appear reddish blue.

FIG. 2 shows the results of generalized analytical 2-D gel map of tNOX isoforms located in quadrant I identified using the diagnostic system of FIG. 1.

FIG. 3 shows the results of generalized analytical 2-D gel map of putative tNOX isoforms located in quadrant IV identified using the diagnostic system of FIG. 1.

FIG. 4 shows the results of analytical 2-D gel electrophoresis and immunoblotting using a tNOX-specific antibody; over quadrant IV from the 2-D gel of serum from a patient with ovarian cancer. The 40.5, 52 and approximately 80 kDa ovarian cancer tNOX isoforms are marked by arrows.

FIG. 5 shows the results of analytical 2-D gel electrophoresis and immunoblotting using a tNOX-specific antibody; as in FIG. 1 except it shows only quadrants I and IV from the same 2-D gel of serum from a patient with small cell lung cancer. These sera contain both 40.5 kDa and 52 kDa tNOX isoforms (arrows), typical of sera from patients with this cancer.

FIG. 6 provides the results of analytical 2-D gel electrophoresis and immunoblotting using tNOX-specific antibody. This figure is as in FIG. 1 except it shows only quadrant I and quadrant IV from the 2-D gel of serum from a patient with non-small cell lung cancer. This serum contains a 54 kDa tNOX isoform characteristic of non-small cell lung cancer (arrow).

FIG. 7 provides the results of analytical 2-D gel electrophoresis and immunoblotting using tNOX-specific antibody. This figure is as in FIG. 1 except it shows only quadrant I and quadrant IV from the 2-D gel of serum from a patient with breast cancer. This serum contains a 68 kDa tNOX isoform, one of the isoforms characteristic of breast cancer (arrow).

FIG. 8 provides the results of analytical 2-D gel electrophoresis and immunoblotting using tNOX-specific antibody. This figure is as in FIG. 1 except it shows only quadrant I from the 2-D gel of serum from a patient with prostate cancer. Plasma and serum from prostate cancer patients contain a characteristic 75 kDa tNOX isoforms (arrows) of isoelectric points of pH 5.8 (a) and pH 5.2 (β) respectively.

FIG. 9 provides the results of analytical 2-D gel electrophoresis and immunoblotting using tNOX-specific antibody. This figure is as in FIG. 1 except it shows only quadrant I of a 2-D gel of serum from a cervical cancer patient. These sera contain a 94 kDa tNOX isoform characteristic of cervical cancer (arrow).

FIG. 10 provides the results of analytical 2-D gel electrophoresis and immunoblotting using tNOX-specific antibody. This figure is as in FIG. 1 except it shows only quadrant I of a 2-D gel of serum from a colon cancer patient. This serum contains tNOX isoforms of 52 and 43 kDa, characteristic of colon cancer (arrows).

FIG. 11 provides the results of analytical 2-D gel electrophoresis and immunoblotting using tNOX-specific antibody. This figure is as in FIG. 1 except it shows only quadrant I and quadrant IV from the 2-D gel of serum from a patient with an unknown primary cancer. This serum contains tNOX isoforms of 40.5, 52 and ca. 80 kDa. The diagnosis based on this analysis is that the primary cancer is of ovarian origin.

FIG. 12 shows results of analytical 2-D gel electrophoresis and immunoblotting using tNOX-specific antibody of sera from a patient with Non-Hodgkins lymphoma illustrating the 43 kDa tNOX isoform of low isoelectric point characteristic of hematological cancers.

FIG. 13 provides the results of analytical 2-D gel electrophoresis and immunoblotting using tNOX-specific antibody. This figure is as in FIG. 1 except it shows only quadrant I and quadrant IV from the 2-D gel of pooled sera from more than 25 random patients with disorders other than cancer, or sera and plasma from healthy volunteers. Both quadrants I and IV are devoid of tNOX isoforms.

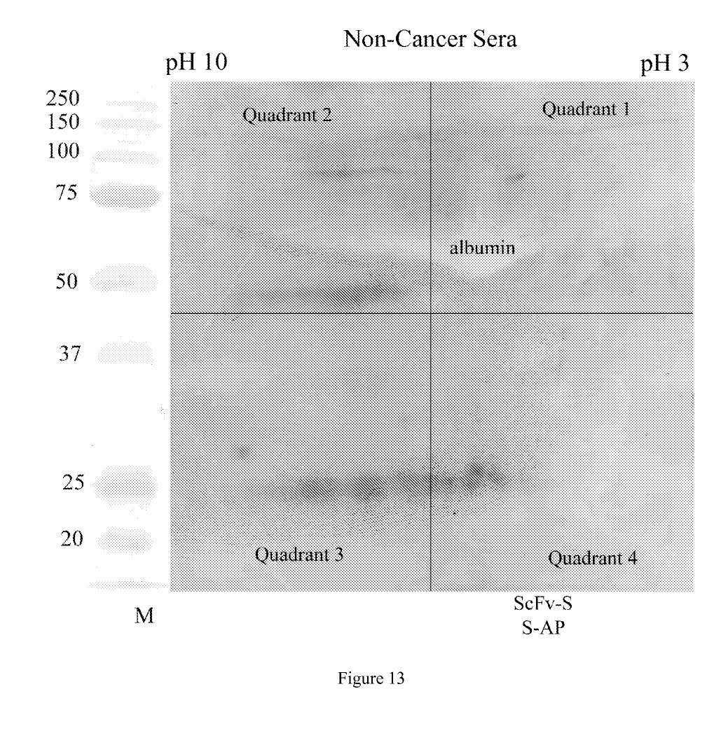

FIG. 14 summarizes the particular tNOX isoforms characteristic of commonly occurring cancers.

### DETAILED DESCRIPTION OF THE INVENTION

The cancer diagnostic system of the present invention utilizes two-dimensional polyacrylamide gel electrophoretic techniques for the separation of proteins in human sera to generate cancer-specific isoform patterns and compositions indicative of cancer presence, tumor type, disease severity and therapeutic response. The protocol is designed for the detection of at least 20 cancer-specific tNOX isoforms which are resolved to indicate cancer presence and disease severity. This specification illustrates the process of the isoform-resolving two-dimensional gel electrophoresis protocol and subsequent immunoanalysis to detect tNOX isoforms which reflect particular cancers.

Two-dimensional gel electrophoresis separates by displacement in two dimensions oriented at right angles to one another and immunoblotting identifies the tNOX isoforms. In the first dimension isoforms are separated according to charge (pI) by isoelectric focusing (IEF). The isoforms are then separated according to size (Mr) by SDS-PAGE in a second dimension. The isoforms are then blotted onto a nitrocellulose membrane for further analysis using a pan-cancer specific antibody preparation.

Rabbits were immunized and sera were prepared against a 33.5 kDa component of culture medium conditioned by the growth of HeLa cells that exhibited an antitumor sulfonylurea-(Morré et al. (1995) Biochim. Biophys. Acta 1240:11-17) and capsaicin-inhibited (Morré et al. (1995) Proc. Natl. Acad. Sci. USA 92:1831-1835) NADH oxidase activity and was capable of binding (³H)-LY181984. These antisera cross-reacted on Western blots with the 34 kDa tNOX protein from HeLa cells and with the 68 kDa and 136 kDa multimeric bands of the material from FPLC separations enriched in capsaicin-inhibitable NADH oxidase activity. No reactivity was observed with preimmune sera.

Antibodies specific for the plasma membrane tNOX and the shed forms in urine and serum of cancer patients and animals with neoplastic disorders are useful, for example, as probes for screening DNA expression libraries or for detecting or diagnosing a neoplastic disorder in a sample from a human or animal. The antibodies (or second antibodies which are specific for the antibody which recognizes tNOX) can be bound to a substance which provides a cofactor, inhibitor, fluorescent agent, chemiluminescent agent, magnetic particle or other detectable signal. Suitable labels include but are not limited to radionuclides, enzymes, substrates, magnetic particles and the like. United States Patents describing the use of such detectable moieties (labels) include, but are not limited to, U.S. Pat. Nos. 3,817,837; 3,850,752; 3,939,350; 3,996,345; 4,277,437; 4,275,149; 4,331,647; 4,348,376; 4,361,544; 5,444,744; 4,460,561; 4,624,846; 4,366,241, 5,716,595; among others. For use in therapeutic regimens, the antibody of the present invention can be coupled to a therapeutic radionuclide, a chemotherapeutic agent, a ribonucleolytic agent or a toxin. See, among others, U.S. Pat. Nos. 5,541,297, 6,395,276.The invention may be further understood by the following non-limiting examples.

Sample preparation is carried out as follows.

Serum tNOX Protein Concentration and Enrichment
1. Add 40 μl of Nickel Agarose Beads to a 0.5 ml Microfuge Tube
   a. Shake beads to disrupt pellet and bring fully into solution
   b. Cut about 2 mm off tip before using
2. Wash Beads
   a. Add 400 μl of deionized distilled-H₂O to fill tubes
   b. Vortex tubes, fully mixing beads for 2-3 seconds
   c. Centrifuge tubes at 1,000 g for 30 seconds
   d. Place tubes vertically in holder allowing beads to settle
   e. Discard water/ethanol supernatant
3. Add 400 μl of Serum to Beads
4. Vortex Tubes for 2-3 Seconds to Fully Disrupt Pellet
5. Incubate Tubes on Horizontal Side at 4° C. on Shaker Overnight

### Protein Purification

1. Centrifuge tubes (above) at 1,000 g for 30 seconds

2. Discard supernatant

3. Wash beads 3x with deionized distilled-H₂0 as described above

First-Dimension (Isoelectric focusing, IEF) is carried out as follows.

### Strip Re-hydration

1. Prepare/thaw re-hydration solution
a. Add 1% dithiothreitol (DTT) to solution immediately before use (0.014 g/1.4 ml)

2. Add 150 μl of re-hydration solution to agarose bound sample

3. Vortex tubes for 75 minutes

4. Remove Immobiline DryStrips (Amersham Pharmacia Biotech) from freezer and allow strips to equilibrate to room temperature for 5 minutes (but not more than 10 min prior to use).
5. Label plastic back of strips with black felt tip pen.
6. Centrifuge sample at 1,000 g for 1 minute to pellet beads.
7. Place tubes in holder vertically and allow pellet to completely settle.
8. Load 125 μl of sample (supernatant) to level tray per 7 cm DryStrip.
9. Place DryStrips gel-side down over sample
10. Insure sample is evenly spread throughout strip by carefully lifting strip in and out of sample a few times if needed.
11. If samples are concentrated in one region of the strip, redistribute sample by pipetting.
12. Remove air bubbles by gently pressing down on DryStrip with pipette tip.
13. Place lid on tray, and place tray in plastic bag with wet paper towels.
14. Seal bag.
15. Allow sample to re-hydrate overnight (between 12-24 hours) at room temperature on level surface allowing strips to absorb samples.

Alternative Sample Preparation.
1. Prepare/thaw re-hydration solution
   a. Add 1% Dithiothreitol (DTT) to solution before use (0.014 g/ 1.4 ml) (contacting with reducing agent)
2. Add 135 μl of re-hydration solution per microfuge tube
3. Add 15 μl of sera per microfuge tube
4. Vortex tubes for 15 min
5. Remove Immobiline DryStrips (Amersham Pharmacia Biotech) from freezer and allow strips to equilibrate to RT for 5 minutes (but not more than 10 min.)
6. Label plastic back of strips
7. Load 125 of sample to level tray per 7 cm. DryStrip.
8. Place DryStrips gel-side down over sample
9. Insure sample is evenly spread throughout strip by carefully lifting strip in and out of sample a few times if needed.
10. If samples are concentrated in one region of the strip, redistribute sample by pipetting.
11. Remove air bubbles by gently pressing down on DryStrip with pipette tip.
12. Place lid on tray, and place tray in plastic bag w/wet paper towels.
13. Seal bag.
14. Allow sample to re-hydrate overnight at RT on level surface allowing strips to absorb samples.
   a. Between 12-24 hours
15. Continue to Isoelectric focusing of strips.

Isoelectric Focusing of Strips
1. Turn on IPGphor 3 (Amersham)
2. Remove strips from tray and carefully remove excess sample from strips by blotting both sides with a tissue.
3. Place strips on manifold focusing tray (Amersham) as follows
   a. Gel side up
   b. Positive (acidic) end towards back
   c. Strips are aligned
   d. Between metal strips (so electrodes fit and touch metal strip)
4. Employ 2 paper wicks (Amersham Pharmacia, #80-6499-14) per strip
5. Wet wicks with 150 μl distilled water per wick.
6. Place wicks over anodic and cathodic ends of gel (approx. 0.3 cm).
7. Place electrodes on wicks, but away from gel (be sure prong is on metal plate), and lock in place.
8. Cover strips, filling entire lane with DryStrip Cover Fluid (Amersham Pharmacia, #17-1335-01)
9. Close apparatus
10. IEF run with IPGphor 3 (Amersham) at a maximum amperage of 50 μAmps at 20° C. As needed, pause run and replace wicks, continuing the run until dye front disappears.

| | Step | Voltage | Time/Vhrs | |
|---|---|---|---|---|
| 7 cm Strip pH 3-10 | 1- | 250 V | 15 | min. |
| | Stp. | | | |
| | 2- | 500 V | 15 | min. |
| | Stp. | | | |
| | 3- | 1000 V | 30 | min. |
| | Stp. | | | |
| | 4- | 4000 V | 2 | hrs. |
| | Grd. | | | |
| | 5- | 4000 V | 25,000 | Vhrs. |
| | Stp. | | | |

| | | |
|---|---|---|
| 6- | 500 V | Hold |
| Stp. | | |

Second-Dimension (SDS-PAGE) is carried out as follows.

Preparation of the SDS-PAGE Gel
1. Prior to use, wash and scrub plates very well in soap and hot water.
2. Rinse in distilled water
3. Leave the plates to air dry or wipe with methanol-soaked tissues.
4. Assemble plates in Protean-plus Multi-Gel Casting Chamber
5. Ensure screws are fully tightened.
6. Degas gel solution prior to addition of APS and TEMED
7. Carefully pour degassed gel solution through gel plates to prevent formation of air bubbles.
8. Stop pouring when gel is about 3 cm from top of glass plates.
9. Gently overlay gels with distilled water.
10. Cover with plastic wrap.
11. Allow gels to polymerize for at least one hour

Equilibration of IEF Strips
1. Remove strips from tray and place on Whatman filter paper to
remove excess oil.
2. Blot both sides of strips with tissue to remove excess oil.
3. Place strips in equilibration plate (Bio-Rad) gel side up; freeze or equilibrate. If frozen, the plates are wrapped in plastic wrap and stored at −80° C. Then, the strips are thawed prior to equilibration (strips are clear when thawed).
   a. Equilibrate: Cover strips with equilibration buffer, about 1.5 ml per strip.
4. Shake 25 minutes at room temperature

Loading and Running Second-Dimension Gel
1. Prepare markers by adding 10 μl of standards on Whatman 3MM chromatography paper cut to approximately 3 cm×0.5 cm.
2. Decant Equilibration Buffer.
3. Cover strips in SDS Running Buffer to rinse away excess Equilibration Buffer.
4. Remove Equilibration Buffer from strips.
5. Repeat steps 3 and 4.
6. Carefully place strips gel side out on back plate of second dimensional gel.
7. Overlay strips with 1% low melting agarose, at room temperature, ensure no air bubbles have formed under the gel.
8. Insert marker next to gel strip's basic end, ensuring marker is flush to gel strip

Agarose must be cool enough to not disrupt marker
9. Allow agarose to solidify
10. Continue for each strip to be loaded in second dimension
11. Place gels in Dodeca tank, hinged side down
12. After all gels have been put in tank, ensure gels are covered entirely by SDS Second dimension electrophoresis is at 13° C., 250 volts for 1 hour 35 minutes.

### Western Blotting and Silver Staining

Transferring Proteins
1. Fill Pyrex tray (large enough to fit gel) with transfer buffer
2. Place sponges in Bio-Rad transblot cell, 2 sponges per gel
3. Fill tank with transfer buffer to allow sponges to saturate with transfer buffer
4. Remove gel from Dodeca tank and cut to desired size
5. Soak pre-cut transfer membrane in transfer buffer
6. Assemble transfer cassette as follows
   a. Black side down
   b. Sponge soaked in transfer buffer
   c. Filter paper
   d. Gel
   e. Nitrocellulose membrane—once placed on gel do not move membrane
   f. Filter paper
   g. Sponge
7. Ensure all air bubbles have been removed between gel and membrane
8. Place tray in Transblot tank, black side (gel side) of tray to black side of tank
9. Transfer at 4° C. at 100 Volts for 60 minutes.

Silver staining to visualize total protein (optional).
1. Put 50 ml of Silver Stain-Fix in container add 25 μl 37% Formaldehyde
2. Put gel in solution and allow to shake for 1 hour to fix proteins
3. Carefully remove Silver Stain-Fix
   a. Only touch gels on edges
4. Cover gels with 50 ml of Silver Stain-Wash and shake for 20 minutes.
5. Remove Silver Stain-Wash solution
6. Add 50 ml. of Silver Stain-Pretreat and shake by hand for 1 minutes.
7. Carefully remove Silver Stain-Pretreat
   a. Only touch gels on edges
8. Carefully rinse gels in distilled water for 20 seconds.
   a. Remove distilled water
   b. Repeat rinse two more times
9. Cover gels with 50 ml of Silver Stain-impregnate and shake for 20 min.
10. Carefully rinse gels in distilled water for 20 seconds.
   a. Remove distilled water
   b. Repeat once
11. Cover gels with 50 ml of Silver Stain-Develop
   c. Shake gels and watch for spots to develop
12. Carefully rinse gels in distilled water for 20 seconds.
   d. Remove distilled water
   e. Repeat rinse two more times
13. Cover gels with 50 ml. of Silver Stain-Fix
14. Shake gels to fix proteins for 10 minutes.

Immunological analysis of the blot is carried out as follows.

### Primary Antibody

1. Remove membrane from transfer
2. Stain membrane with Ponceau-S until background is red.
3. Remove Ponceau-S (return to bottle for reuse)
4. Rinse membrane with water and label.
5. Scan membrane
6. Wash membrane with TTBS to remove all Ponceau-S
7. Add 5% milk (enough to cover membrane) to tray and block for 20 minutes at room temperature
8. Prepare primary antibody solution (NTI ScFv MAB 12.1®:TTBS-1:150) in container
9. Remove milk
10. Rinse to remove excess milk in TTBS
11. Place membrane into container with primary antibody solution
12. Incubate at 4° C. overnight

### Secondary Antibody

1. Remove Primary antibody
2. Wash membrane 3 times
   i. Cover membrane with TTBS
   ii. Gently shake at room temperature for 10 minutes.
3. Prepare secondary antibody solution (NTI Anti S:TTBS-1:5,000)
4. Cover membrane with secondary antibody solution
5. Incubate at 4° C. for 4 hours.

### Develop and Scan

1. Remove secondary antibody solution
2. Wash membrane as above
3. Cover membrane with Western Blue (Promega, #S3841)
4. Allow to develop
5. Stop develop by rinsing in water
6. Dry membrane

Solutions used in the foregoing protocols are given below.

### Solutions for First-Dimension

### Rehydration Buffer pH 7 (25 mL):

7 M Urea (FW 60.06)-10.5 g
2 M Thiourea (FW 76.12)-3.8 g
2% CHAPS-0.5 g
0.5% asb-14-0.125 g
0.5% Ampholytes-330 μl (40% Ampholytes)
0.5% IPG Buffer-125 μl
Bromophenol Blue-3 mg
   Dissolve Urea & Thiourea in 20 ml of distilled water.
Add CHAPS, AsB-14, Ampholytes, IPG Buffer and Bromophenol Blue
Fill to 25 ml with distilled water
Aliquot to 1.4 ml and store at −80° C.
Add 1% DTT-14 mg (0.014 g) before use

### Solutions for Second-Dimension

### Tris Buffer (1.5 M, pH 8.8) (1000 mL):

Tris-base (FW 121.1)-36.33 g
Dissolve Tris in about 150 mL distilled water.
adjust pH to 8.8 with HCl, and make up to a total volume of 200 ml with distilled water.
store at 4° C.

### Equilibration Buffer (400 mL):

1.5 M Tris-HCL, pH 8.8-26.8 mL
Urea (60.06)-144 g
Glycerol (100%)-120 mL (150 g)
SDS-10 g
   Bromophenol Blue-Trace (add with pipette tip)
Add water to bring to 400 mL
Aliquot and store at −20° C.

### 2D Acrylamide Gel

375 mM Tris-HCl, pH 8.8
Acrylamide/Piperazine Diacrylyl (40% T/2.5% C)
0.12% (v/v) TEMED
0.055% (v/v) ammonium persulfate (APS)
   %T=Total percent of Acrylamide and Piperazine Diacrylyl in the 2D Acrylamide Gel

### %C =Percentage of the Cross-linker (Piperazine Diacrylyl)

Formulations for Protean II gels (20×20; 1-mm thick)

| | | Gel Buffer (ml) | 40% Acrylamide (ml) w/ | | 10% APS | |
|---|---|---|---|---|---|---|
| % | Gels | (1.875 M Tris-HCl) | PDA (39:1) | Water (ml) | (ml) | TEMED (μl) |
| 10 | 2 | 30 | 37.5 | 82.5 | 0.825 | 235 |
| | 4 | 50 | 62.5 | 137.5 | 1.375 | 340 |
| | 6 | 70 | 87.5 | 192.5 | 1.925 | 545 |
| | 8 | 90 | 112.5 | 247.5 | 2.5 | 700 |
| | 10 | 110 | 137.5 | 302.5 | 3.025 | 855 |
| | 12 | 130 | 162.5 | 357.5 | 3.575 | 1000 |

Gel Buffer

| Reagents | Amount |
|---|---|
| 1.875 M Tris | 22.7 g |
| water | Fill to 100 ml |
| adjust pH to | use concentrated |
| 8.8 | HCl |

Store the buffer at 4° C. for no more than 2 weeks.

Acrylamide Stock - 40% T/2.5% C.

| Reagents | Amount |
|---|---|
| acrylamide | 39 g |
| piperazine | 1 g |
| diacrylamide | |
| water | Fill to 100 ml |

Filter (0.45 μm), store at 4° C. for no more than 2 weeks.

10% Ammonium Persulfate (APS)

| Reagents | Amount |
|---|---|
| 10% APS | 0.1 g |
| water | Fill to 1 ml |

Make 10% Ammonium Persulfate fresh for every use

10x SDS Running Buffer (4 L):

| | |
|---|---|
| Tris (FW 75.07) | 75.69 g |
| Glycine (FW 121.14) | 360.34 g |
| SDS | 20 g |
| Distilled water | Fill to 4 L |

Store at room temperature

Agarose Solution (1%):
Weigh 1.5 g of agarose, add 150 mL SDS Running Buffer -
heat until it dissolves.

| | |
|---|---|
| 1% Low Melt Agarose | 1.5 g Low Melt Agarose |
| 1x SDS Run Buffer | Fill to 150 ml |

Heat until agarose boils and fully dissolves
Add 150 μl of 0.05% Bromophenol blue
Stored at 4° C.

### Solutions for Blotting and Silver Staining

Western Transfer Buffer (4 L):

| | |
|---|---|
| Trizma base | 12.12 g |
| Glycine | 57.6 g |
| Methanol | 800 ml |
| SDS | 3 g |
| Water | Fill to 4 L |

Silver Stain-Fix

| | |
|---|---|
| 50% methanol | 500 ml |
| 12% acetic acid | 120 ml |
| distilled water | fill to 1 L |

Silver Stain-Ethanol

| | |
|---|---|
| 50% ethanol | 500 ml |
| distilled water | fill to 1 L |

Silver Stain-Pretreat

| | |
|---|---|
| 0.04% Na₂S₂O₃—5H₂O | 0.04 g |
| distilled water | fill to 200 ml |

Silver Stain-Impregnate

| | |
|---|---|
| 0.4% AgNO₃ | 0.4 g |
| 0.028% formaldehyde | 150 μl 37% formaldehyde |
| distilled water | fill to 200 ml |

Silver Stain-Develop

| | |
|---|---|
| 12% Na₂CO₃ | 12 g |
| 0.019% formaldehyde | 100 μl 37% formaldehyde |
| 2% Pretreat | 4 ml |
| distilled water | fill to 200 ml |

37% Formaldehyde

| | |
|---|---|
| 37% formaldehyde | 37 ml formaldehyde |
| distilled water | fill to 100 ml |

Blocking Buffer (5% milk)

| | |
|---|---|
| 5% milk | 5 g Milk |
| 0.2% N₃Na | 0.2 g N₃Na |
| distilled water | fill to 100 ml |

10x TTBS (4 L)

| | |
|---|---|
| 100 mM Trizma | 48.4 g |
| 1.5 M NaCl | 350.6 g |
| 0.5% Tween 20 | 20 g |

Add Trizma and NaCl to 3800 ml deionized distilled-H₂O
Adjust pH to 8.0 using HCl
Add Tween 20
Fill to 4 L with distilled water

### EXAMPLES

### Example 1. Pooled Sera

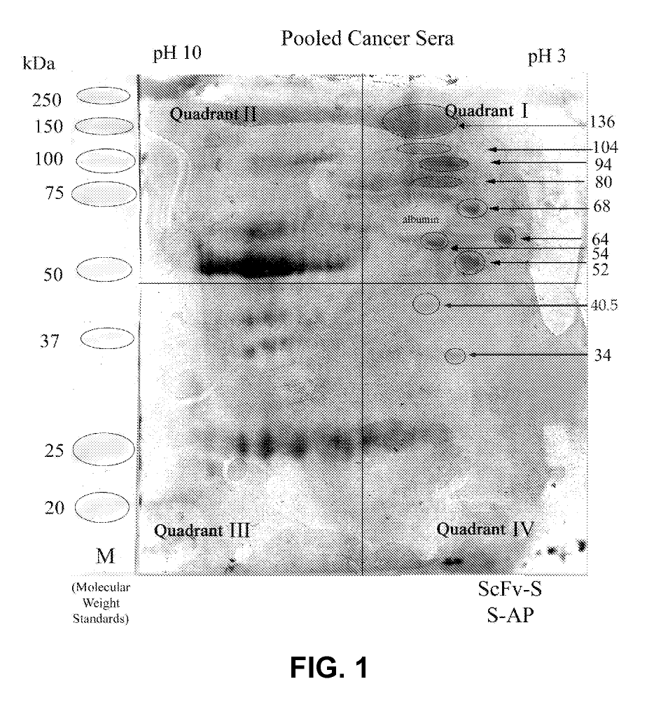

NOX-Enriched serum proteins (approximately 4-6 mg) from sera pooled from cancer patients (breast, ovarian, lung and colon) were resolved by 2-D gel electrophoresis with detection by recombinant anti-ECTO-NOX antibody (single chain variable region ScFv) carrying an S-tag followed by alkaline phosphatase-linked anti-S with Western Blue NBT alkaline phosphatase substrate yield several proteins present in the cancer sera (FIG. 1) but absent from sera of non-cancer patients or healthy volunteers. The tNOX proteins were enriched and concentrated from the sera by nickel agarose precipitation. This step results in removal of approximately 90% of serum proteins prior to analysis.

Where sera specific for tNOX or ECTO-NOX are used, typically the dilution is from 1:10 to 1:1000, monoclonal antibody (e.g., 12.1 produced by hybridoma on deposit as ATCC Accession No. PTA-4206) is from 1 ;100-1:10,00, desirably about 1:100, or ascites fluid from growth of the same hybridoma can be from about 1:10 to about 1:1000, desirably about 1:100.

### Example 2. Analysis of Sera from Patients with Various Cancers

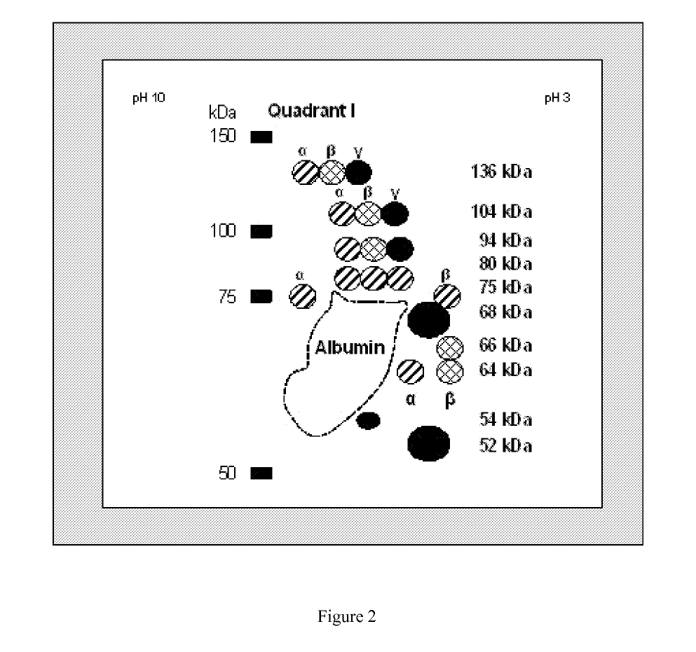

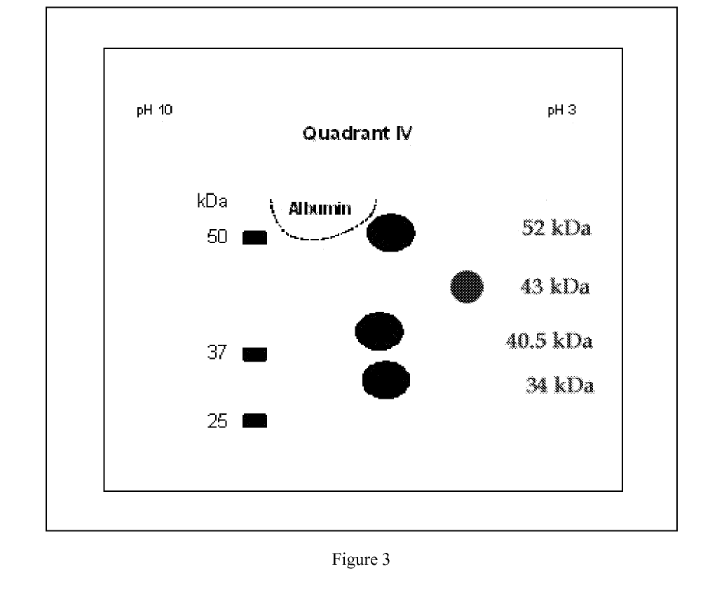

Results combined from a total of eight different pooled cancer sera were similar to those of FIG. 1 and the total of the findings are summarized in FIG. 2 for quadrant I of the 2-D gels. Some additional tNOX proteins were located in quadrant IV and the total of the findings are summarized in FIG. 3.

### Example 3. Analysis of Ovarian Cancer Patient Serum

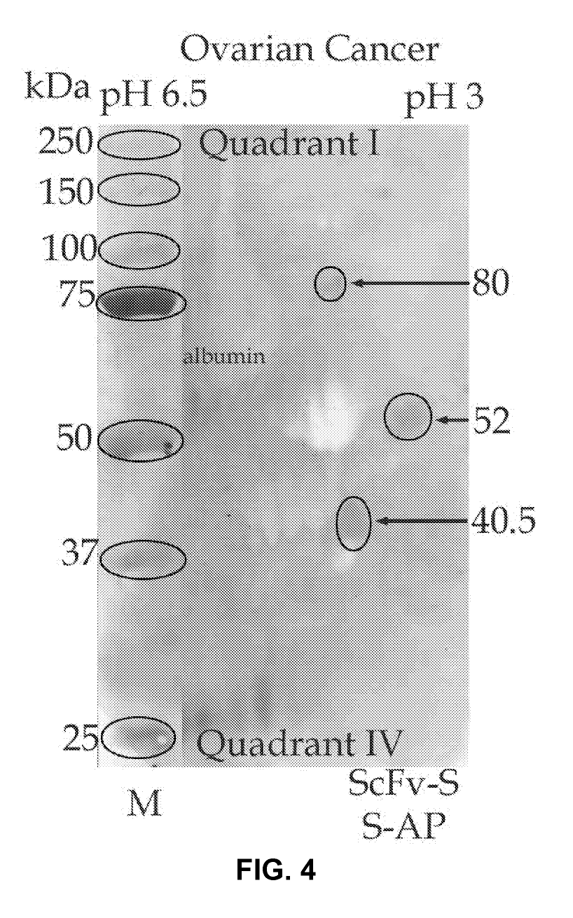

2-D gel analysis as in FIG. 4 when applied to serum from a patient with ovarian cancer yielded ovarian cancer specific tNOX proteins of about 80 and 40.5 kDa in quadrants I and IV (FIG. 4).

### Example 4. Analysis of Small Cell Lung Cancer Patient Serum

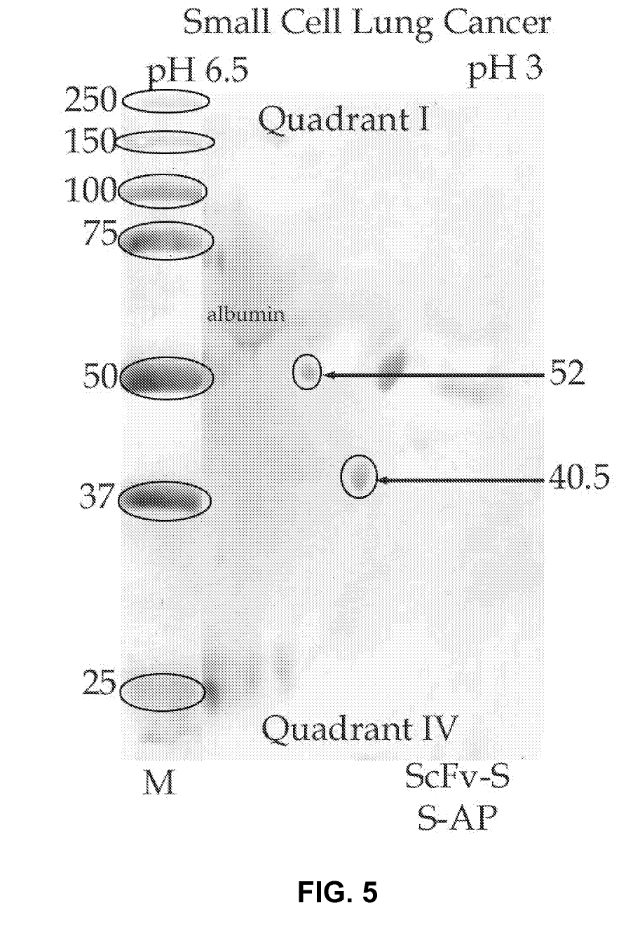

2-D gel analysis as in FIG. 5 when applied to sera of a patient with small cell lung cancer contained a 40.5 kDa tNOX protein in quadrant IV and a 52 kDa tNOX protein in quadrant I (FIG. 5).

### Example 5. Analysis of Non Small Cell Lung Cancer Patient Serum

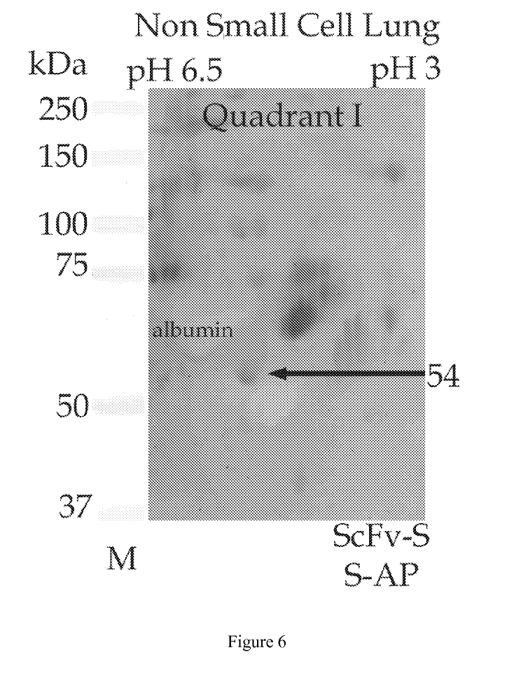

2-D gel analysis as in FIG. 1 when applied to plasma from a patient with non-small cell lung cancer revealed a non-small cell cancer specific tNOX isoform at 54 kDa in quadrant 1 (FIG. 6).

### Example 6. Analysis of Breast Cancer Patient Serum

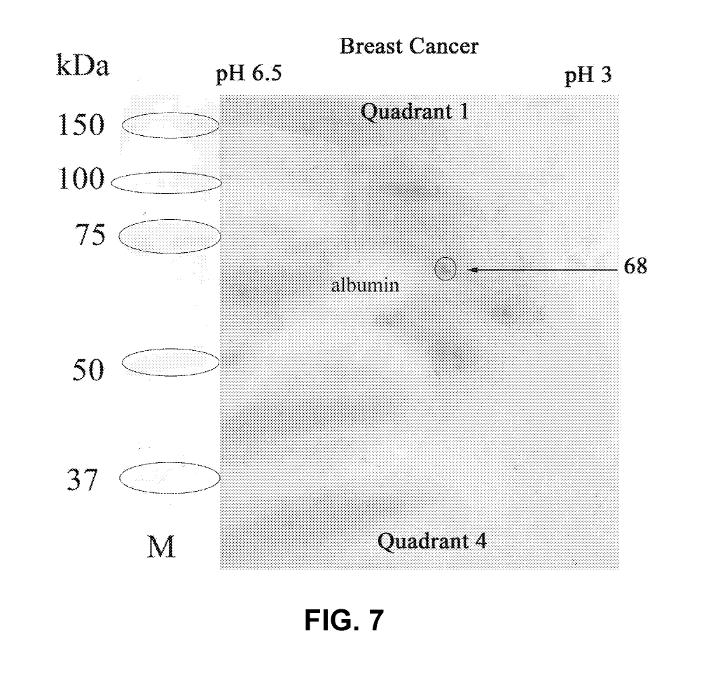

2-D gel analysis as in FIG. 1 when applied to sera from a patient with breast cancer revealed a breast cancer-specific tNOX protein of 68 kDa in quadrant I (FIG. 7).

### Example 7. Analysis of Prostate Cancer Patient Serum

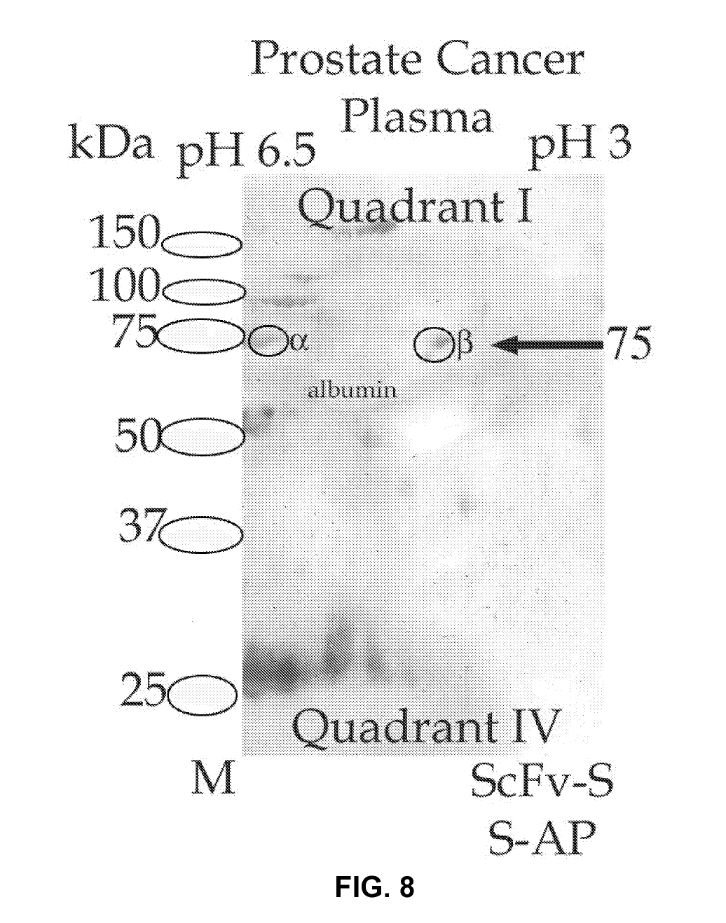

2-D gel analysis as in FIG. 1 when applied to sera from a patient with prostate cancer revealed prostate cancer-specific tNOX isoforms at 75 kDa (FIG. 8) ) and isoelectric points of pH 5.8 (α) and 5.2 (β). The β-form is most evident with plasma and present infrequently in sera of prostate cancer patients.

### Example 8. Analysis of Cervical Cancer Patient Serum

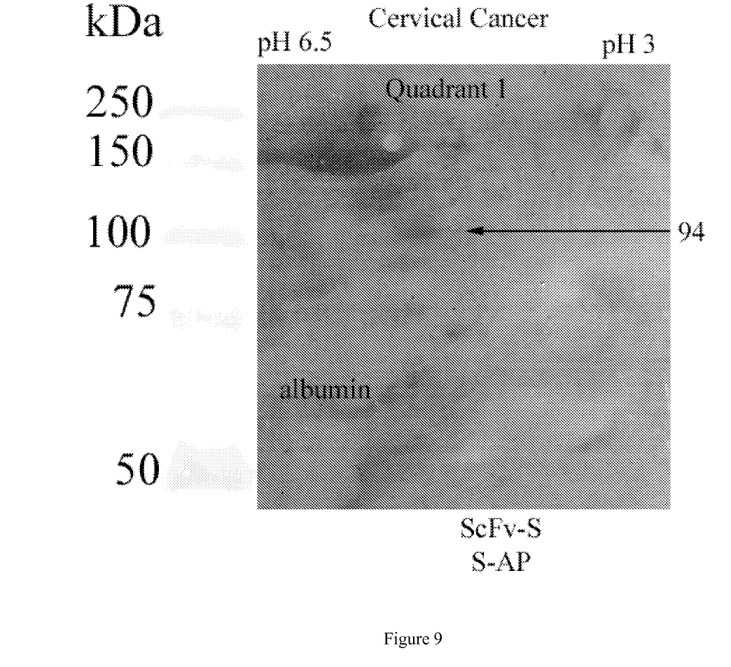

2-D gel analysis as in FIG. 1 when applied to serum from a patient with cervical cancer revealed a cervical cancer-specific tNOX isoform at 94 kDa (FIG. 9).

### Example 9. Analysis of Colon Cancer Patient Serum

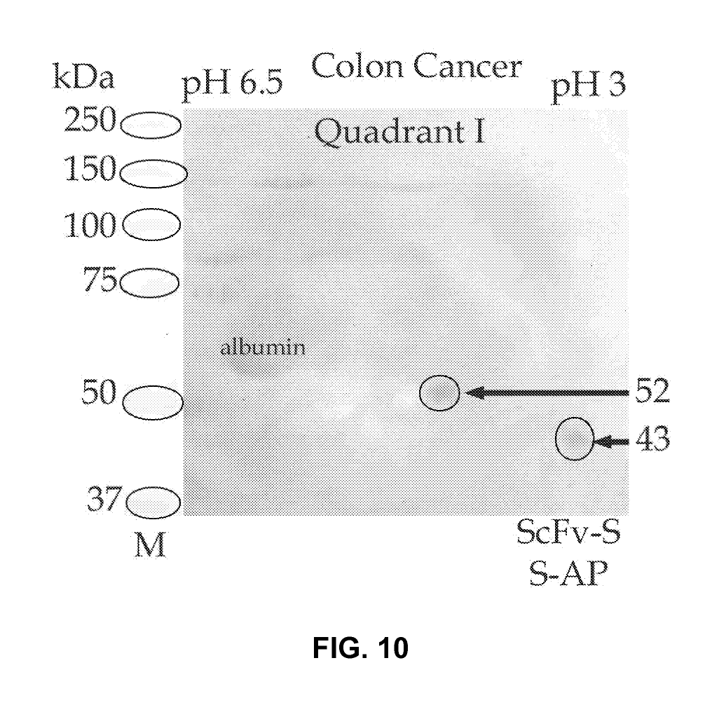

2-D gel analysis as in FIG. 1 when applied to serum from a patient with colon cancer revealed colon cancer-specific tNOX isoforms at 43 and 52 kDa (FIG. 10).

### Example 10. Cancer Specific Isoforms

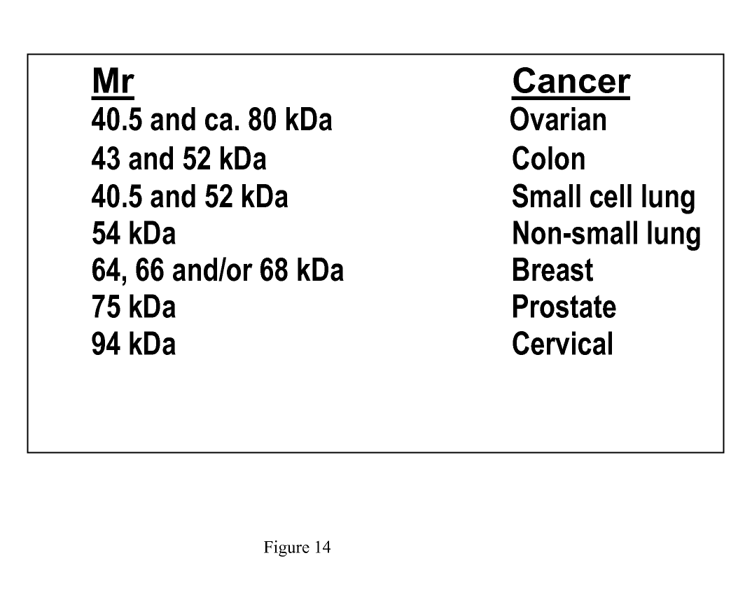

For each kind of cancer there appears to be a tNOX isoform (ovarian, breast, cervical, colon, non-small cell lung, prostate small cell lung) or combination of tNOX isoforms that is specific to the tissue or cell type of origin for the cancer (FIG. 14).

### Example 11. Analysis of Patient Serum where Cancer of Unknown Origin

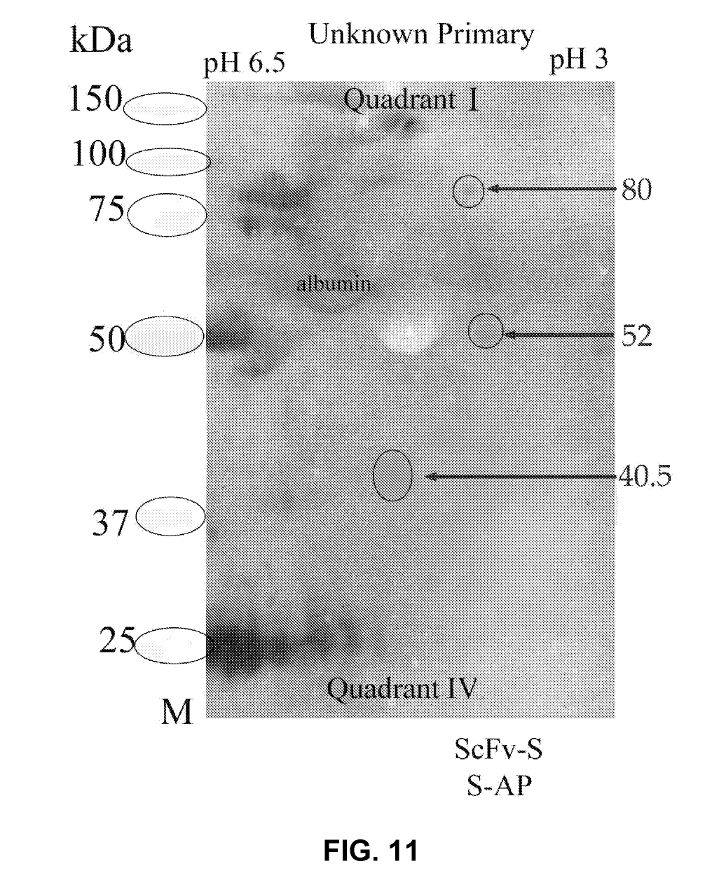

The 2-D gel of FIG. 11 is from a patient with cancer where the primary tumor was unknown. The presence of 40.5, 52 and ca. 80 kDa tNOX isoforms indicates the primary cancer is ovarian cancer.

### Example 12. Analysis of Pooled Normal Sera

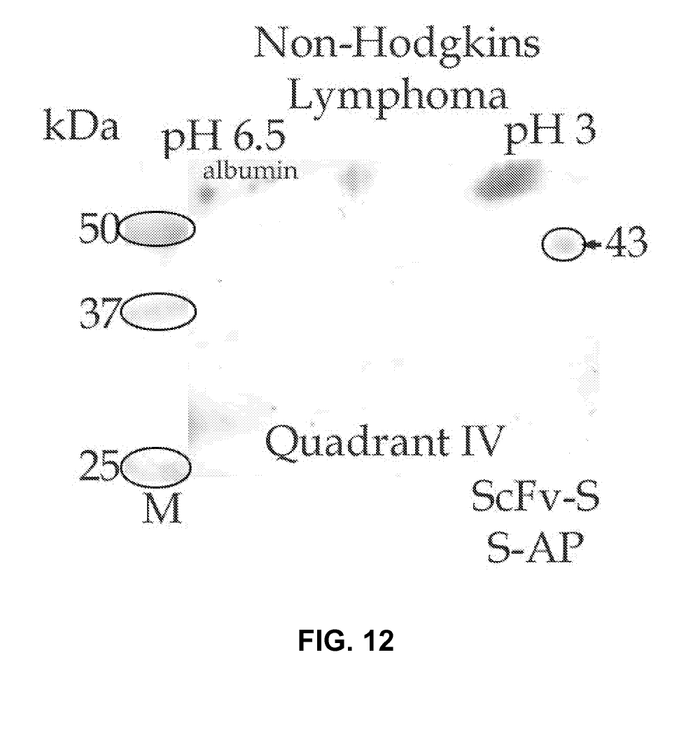

In more than 25 randomly selected outpatient sera and sera of healthy volunteers, both quadrants I and IV of the 2-D gels were devoid of tNOX isoforms (FIG. 12), confirming previous observations that tNOX proteins are absent from non-cancer patients or sera of healthy volunteers.

### Example 13.

The diagnostic strategy of the invention combines one- and two-dimensional polyacrylamide gel electrophoretic separations of human sera to generate cancer specific isoform patterns and compositions indicative of cancer presence, tumor type, disease severity and therapeutic response. At least 20 cancer-specific tNOX isoforms are resolved indicative of cancer presence and disease severity. The system consists of a concentration step where ECTO-NOX proteins are separated from the bulk of the albumin and other serum proteins using a nickel-agarose precipitation. Detection uses a recombinant single chain antibody (scFv) that cross reacts with all known ECTO-NOX isoforms of human origin; this antibody also has an S-tag that reacts with a second antibody I (anti-S). The scFv antibody is described in U.S. Provisional Application 60/824,398, filed Sep. 1, 2006 and below.

Monoclonal antibody generated against tNOX NADH oxidase tumor cell specific was produced in sp-2 myeloma cells; however, the monoclonal antibody slowed the growth of sp-2 myeloma cells that were used for fusion with spleen cells after 72 h. This phenomenon made it difficult to produce antibody in quantity. To overcome this problem, the coding sequences of the antigen-binding variable region of the heavy chain and the light chain (Fv region) of the antibody cDNA were cloned and linked into one chimeric gene, upstream of the S-tag coding sequence. The Fv portion of an antibody, consisting of variable heavy (V_{H}) and variable light (V_{L}) domains, can maintain the binding specificity and affinity of the original antibody (Glockshuber et al. 1990. Biochemistry 29:1262-1367).

For a recombinant antibody, cDNAs encoding the variable regions of immunoglobulin heavy chain (V_{H}) and light chain (V_{I}), are cloned by using degenerative primers. Mammalian immunoglobulins of light and heavy chain contain conserved regions adjacent to the hypervariable complementary defining regions (CDRs). Degenerate oligoprimer sets allow these regions to be amplified using PCR (Jones et al. 1991. BiolTechnology 9:88-89; Daugherty et al. 1991. Nucleic Acids Research 19:2471-2476). Recombinant DNA techniques have facilitated the stabilization of variable fragments by covalently linking the two fragments by a polypeptide linker (Huston et al. 1988. Proc. Natl. Acad. Sci. USA 85:5879-5883). Either V_{L} or V_{H} can provide the NH₂-terminal domain of the single chain variable fragment (ScFv). The linker should be designed to resist proteolysis and to minimize protein aggregation. Linker length and sequences contribute and control flexibility and interaction with ScFv and antigen. The most widely used linkers have sequences consisting of glycine (Gly) and serine (Ser) residues for flexibility, with charged residues as glutamic acid (Glu) and lysine (Lys) for solubility (Bird et al. 1988. Science 242:423-426; Huston et al. 1988. supra).

Total RNA was isolated from the hybridoma cells producing tNOX-specific monoclonal antibodies by the following procedure modified from Chomczynski et al. (1987) Anal. Biochem. 162:156-159 and Gough (1988) Anal. Biochem 176:93-95. Cells were harvested from medium and pelleted by centrifugation at 450× g for 10 min. Pellets were gently resuspended with 10 volumes of ice cold PBS and centrifuged again. The supernatant was discarded and cells were resuspended with an equal volume of PBS. Denaturing solution (0.36 ml of 2-mercaptoethanol/50 ml of guanidinium stock solution-4M guanidinium thiocyanate, 25 mM sodium citrate, pH 7.0, 0.5% sarkosyl) 10 ml per 1 g of cell pellet was added prior to use and mixed gently. Sodium acetate (pH 4.0,1 ml of 2M), 10 ml of phenol saturated water and 2 ml of chloroform: isoamyl alcohol (24:1) mixtures were sequentially added after each addition. The solution was mixed thoroughly by inversion. The solution was vigorously shaken for 10 sec, chilled on ice for 15 min and then centrifuged 12,000× g for 30 min. The supernatant was transferred and an equal volume of 2-propanol was added and placed at −20° C. overnight to precipitate the RNA. The RNA was pelleted for 15 min at 12,000× g, and the pellet was resuspended with 2-3ml of denaturing solution and 2 volumes of ethanol. The solution was placed at −20° C. for 2 h, and then centrifuged at 12,000× g for 15 min. The RNA pellet was washed with 70% ethanol and then 100% ethanol. The pellet was resuspended with RNase-free water (DEPC-treated water) after centrifugation at 12,000× g for 5 min. The amount of isolated RNA was measured spectrophotometrically and calculated from the absorbance at 280 nm and 260 nm.

The poly(A)mRNA isolation kit was purchased from Stratagene. Total RNA was applied to an oligo(dT) cellulose column after heating the total RNA at 65° C. for 5 min. Before applying, the RNA samples were mixed with 500 μl of 10× sample buffer (10 mM Tris-HCl, pH 7.5, 1 mM EDTA, 5 M NaCl). The RNA samples were pushed through the column at a rate of 1 drop every 2 sec. The eluates were pooled and reapplied to the column and purified again. Preheated elution buffer (65° C.) was applied, and mRNA was eluted and collected in 1.5 ml of centrifuge tubes on ice. The amount of mRNA was determined at OD₂₆₀ (1 OD unit=40 μg of RNA). The amounts of total RNA and mRNA obtained from 4×10⁸ cells were 1328 μg and 28 μg, respectively.

mRNA (1-2 μg) dissolved in DEPC-treated water was used for cDNA synthesis. mRNA isolated on three different dates was pooled for first-strand cDNA synthesis. The cDNA synthesis kit was purchased from Pharmacia Biotech. mRNA (1.5 μg/5 μl of DEPC-treated water) was heated at 65° C. for 10 min. and cooled immediately on ice. The primed first strand mix containing MuLV reverse transcriptase (11 μl) and appropriate buffers for the reaction were mixed with mRNA sample. DTT solution (1 μl of 0.1 M) and RNase-free water (16 μl) also were added to the solution. The mixture was incubated for 1 h at 37° C.

Degenerate primers for light chain and heavy chain (Novagen, Madison, Wis.) were used for PCR. PCR synthesis was carried out in 100 μl reaction volumes in 0.5 ml microcentrifuge tubes by using Robocycler (Stratagene, La Jolla, Calif.). All PCR syntheses included 2 μl of sense and anti-sense primers (20 pmoles/μl), 1 μl of first-strand cDNA as a template, 2 μl of 10 mM of dNTPs, 1 μl of Vent polymerase (2 units/μl), 10 μl of 10×PCR buffer (100 mM Tris-HCl, pH 8.8 at 25° C., 500 mM KCl, 15 mM MgCl₂, 1% Triton X-100), 82 μl of H₂O. Triton X-100 is t-octylphenoxypolyethoxyethanol. All PCR profiles consisted of 1 min of denaturation at 94° C., 1 min of annealing at 55° C., and 1 min of extension at 72° C. This sequence was repeated 30 times with a 6-min extension at 72° C. in the final cycle. PCR products were purified with QIAEX II gel extraction kit from Qiagen, Valencia, Calif. PCR amplification products for heavy and light chain coding sequences were analyzed by agarose gel electrophoresis and were about 340 base pair (bp) long and 325 bp long, respectively.

Total RNA or DNA was analyzed by agarose gel electrophoresis (1% agarose gels). Agarose (0.5 g in 50 ml of TAE buffer, 40 mM Tris-acetate, 1 mM EDTA) was heated for 2 min in a microwave to melt and evenly disperse the agarose. The solution was cooled at room temperature, and ethidium bromide (0.5 μg/ml) was added and poured into the apparatus. Each sample was mixed with 6×gel loading buffer (0.25% bromophenol blue, 0.25% xylene cyanol FF, 40% (w/v) sucrose in water). TAE buffer was used as the running buffer. Voltage (10 v/cm) was applied for 60-90 min.

According to the proper size for heavy and light chain cDNAs, the bands were excised from the gels under UV illumination, and excised gels were placed in 1 ml syringes fitted with 18-gauge needles. Gels were crushed to a 1.5 ml Eppendorf tube. The barrel of each syringe was washed with 200 μl of buffer-saturated phenol (pH 7.9±0.2). The mixture was thoroughly centrifuged and frozen at −70° C. for 10 min. The mixture was centrifuged for 5 min, and the top aqueous phase was transferred to a new tube. The aqueous phase was extracted again with phenol/chloroform (1:1). After centrifuging for 5 min, the top aqueous phase was transferred to a clean tube, and chloroform extraction was performed. Sodium acetate (10 volumes of 3 M) and 2.5 volumes of ice-cold ethanol were added to the top aqueous phase to precipitate DNA at −20° C. overnight.

Purified heavy and light chain cDNAs were ligated into plasmid pSTBlue-1 vector and transfected into NovaBlue competent cells (Stratagene). Colonies containing heavy and light chain DNAs were screened by blue and white colony selection and confirmed by PCR analysis. Heavy and light chain DNAs were isolated and sequenced using standard techniques. Tables 2A and 2B show the DNA sequences of heavy and light chain DNAs of ScFv. See also SEQ ID NO: 3 and SEQ ID NO: 4.

PCR amplification and the assembly of single ScFv gene was according to Davis et al. (1991) Bio/Technology9:165-169. Plasmid pSTBlue-1 carrying V_{H} and V_{L} genes were combined with all four oligonucleotide primers in a single PCR synthesis. Following first PCR synthesis, one tenth of the first PCR product was removed and added to a second PCR reaction mixture containing only the primer a (V_{H} sense primer) and primer d (V_{L} Antisense primer). The product of the second PCR synthesis yielded single ScFv gene. The single ScFv gene was ligated to plasmid pT-Adv (Clontech, Palo Alto, Calif.). pT-Adv carrying ScFv gene was used for DNA sequencing.

The complete ScFv gene was assembled from the V_{H}, V_{L} and linker genes to yield a single ScFv gene by PCR (Tables 2A and 2B). The DNA sequence encoding the linker was 45 nucleotides long (GGAGGCGGTGGATCGGGCGGTGGCGGCTCGGGTGGCGGCGGCTCT; SEQ ID NO:6), which translates to a peptide of 15 amino acids (GlyGlyGlyGlySerGlyGlyGlyGlySerGlyGlyGlyGlySer; SEQ ID NO:5). Primers for PCR amplification are shown in Tables 2A and 2B. S-peptide was linked to the C-terminus of ScFv[ScFv(S)]. S-peptide binds to S-protein conjugated to alkaline phosphatase for Western blot analysis. The DNA sequence of the S-peptide is AAAGAAACCGCTGCTGCTAAATTCGAACGCCAGCACATGGACAGC (SEQ ID N 0:7) which translates to S-peptide (LysGluThrAlaAlaAlaLysPheGluArgGlnHisMetAspSer; SEQ ID NO:8).

Recombinant ScFv(S) was expressed in E. coli. First, oligo nucleotides encoding S-peptide were linked to the 3′ end of the open reading frame (ORF) of ScFv DNA by PCR amplification. Incorporation of S-peptide enables to detect expressed ScFv protein by S-protein conjugated to alkaline phosphatase. The tNOX-specific ScFv(S) coding sequence was then subcloned to plasmid pET-11a, a plasmid designed for protein expression in E. coli (Stratagene, Calif.). For PCR amplification, two primers were designed to amplify ORF of ScFv(S) containing endonuclease restriction sites (Ndel and Nhel) and S-peptide residues. Plasmid pET-11a and ORF of ScFv(S) were digested with restriction enzymes Ndel and Nhel and ligated to produce plasmid pET11-ScFv(S). E. coli BL21 (DE3) was transformed with pET11-ScFv(S) and grown at 37° C. for 12 h in LB medium containing ampicillin (100 μg/ml). ScFv was expressed by addition of 0.5 mM IPTG and incubation for 4 h. Cells were harvested and lysed using a French Pressure Cell (French Pressure Cell Press, SLM Instruments, Inc.) (three passages at 20,000 psi). Cell extracts were centrifuged at 10,000×g for 20 min. Pellets containing denatured inclusion bodies of ScFv were collected. Renaturation of the inclusion bodies of the ScFv was according to Goldberg et al. (1995) Folding & Design 1:21-27.

TABLE 1A
DNA Sequence of Heavy Chain ScFv (V_{H}), SEQ ID NO: 1

| | |
|---|---|
| 1 | gaggtcaagc tgcaggagtc aggaactgaa gtggtaaagc ctggggcttc |
| 51 | agtgaagttg tcctgcaagg cttctggcta catcttcaca agttatgata |
| 101 | tagactgggt gaggcagacg cctgaacagg gacttgagtg gattggatgg |
| 151 | atttttcctg gagaggggag tactgaatac aatgagaagt tcaagggcag |
| 201 | ggccacactg agtgtagaca agtcctccag cacagcctat atggagctca |
| 251 | ctaggctgac atctgaggac tctgctgtct atttctgtgc tagaggggac |
| 301 | tactataggc gctactttga cttgtggggc caagggacca cggtcaccgt |
| 351 | ctcctca |

TABLE 1B
DNA sequence of light chain ScFv (V_{L}), SEQ ID NO: 2

| | |
|---|---|
| 1 | gaaaatgtgc tcacccagtc tccagcaatc atgtctgcat ctccagggga |
| 51 | gagggtcacc atgacctgca gtgccagctc aagtatacgt tacatatatt |
| 101 | ggtaccaaca gaagcctgga tcctccccca gactcctgat ttatgacaca |
| 151 | tccaacgtgg ctcctggagt cccttttcgc ttcagtggca gtgggtctgg |
| 201 | gacctcttat tctctcacaa tcaaccgaat ggaggctgag gatgctgcca |
| 251 | cttattactg ccaggagtgg agtggttatc cgtacacgtt cggagggggg |
| 301 | accaagctgg agctgaaagc g |

TABLE 2A
Coding Sequence for ScFv, SEQ ID NO: 3

| | |
|---|---|
| 1 | gaggtcaagc tgcaggagtc aggaactgaa gtggtaaagc ctggggcttc |
| 51 | agtgaagttg tcctgcaagg cttctggcta catcttcaca agttatgata |
| 101 | tagactgggt gaggcagacg cctgaacagg gacttgagtg gattggatgg |
| 151 | atttttcctg gagaggggag tactgaatac aatgagaagt tcaagggcag |
| 201 | ggccacactg agtgtagaca agtcctccag cacagcctat atggagctca |
| 251 | ctaggctgac atctgaggac tctgctgtct atttctgtgc tagaggggac |
| 301 | tactataggc gctactttga cttgtggggc caagggacca cggtcaccgt |
| 351 | ctcctcagga ggcggtggat cgggcggtgg cggctcgggt ggcggcggct |
| | linker |
| 401 | ctgaaaatgt gctcacccag tctccagcaa tcatgtctgc atctccaggg |
| 451 | gagagggtca ccatgacctg cagtgccagc tcaagtatac gttacatata |
| 501 | ttggtaccaa cagaagcctg gatcctcccc cagactcctg atttatgaca |
| 551 | catccaacgt ggctcctgga gtcccttttc gcttcagtgg cagtgggtct |
| 601 | gggacctctt attctctcac aatcaaccga atggaggctg aggatgctgc |
| 651 | cacttattac tgccaggagt ggagtggtta tccgtacacg ttcggagggg |
| 701 | ggaccaagct ggagctgaaa gcgaaagaaa ccgctgctgc taaattcgaa |
| 751 | cgccagcaca tggacagc s-peptide |

TABLE 2B
Amino Acid Sequence for ScFv, SEQ ID NO: 4

| | |
|---|---|
| 1 | EVKLQESGTE VVKPGASVKL SCKASGYIFT SYDIDWVRQT PEQGLEWIGW |
| 51 | IFPGEGSTEY NEKFKGRATL SVDKSSSTAY MELTRLTSED SAVYFCARGD |
| 101 | YYRRYFDLWG QGTTVTVSSG GGGSGGGGSG GGGSENVLTQ SPAIMSASPG |
| | linker |
| 151 | ERVTMTCSAS SSIRYIYWYQ QKPGSSPRLL IYDTSNVAPG VPFRFSGSGS |
| 201 | GTSYSLTINR MEAEDAATYY CQEWSGYPYT FGGGTKLELK AKETAAAKFE |
| 251 | RQHMDS s-peptide |

TABLE 3
Primers for PCR amplification of ScFv(s) gene

1. Primers for cloning of variable regions of heavy chain and light chain
of antibody
(A) Primers for heavy chain (V_{H})

| | | |
|---|---|---|
| Forward primer: | 5′-GGCCCAGCCGGCCGAGGTCAAGCTGCAGGAGTCAGGA- 3′ | (SEQ ID NO: 9) |
| Reverse primer: | 5′-CTCGGAACCTGAGGAGACGGTGACCGTGGTCCC- 3′ | (SEQ ID NO: 10) |

(B) Primers for light chain (V_{L})

| | | |
|---|---|---|
| Forward primer: | 5′-TCCAAAGTCGACGAAAATGTGCTCACCCAGTCTCCA- 3′ | (SEQ ID NO: 11) |
| Reverse primer: | 5′-AGCGGCCGCTTTCAGCTCCAGCTTGGTCCCCCC- 3′ | (SEQ ID NO: 12) |

2. Primers for subcloning of ScFv(s) gene into pET-11a expression vector
(A) Primers for heavy chain (V_{H}) and linker amplification

| | | |
|---|---|---|
| Forward primer: | 5′-GTCAAGCTGCAGGAGTCAGGA- 3′ | (SEQ ID NO: 13) |
| Reverse primer: | 5′-AGAGCCGCCGCCACCCGAGCCGCCACCGCCCGATCC | (SEQ ID NO: 14) |
| | ACCGCCTCCTGAGGAGACGGTGACCGTGGT- 3′ | |

(B) Primers for light chain (V_{L}), linker and S-tag amplification

| | | |
|---|---|---|
| Forward primer: | 5′-GGAGGCGGTGGATCGGGCGGTGGCGGCTCGGGTGGC | (SEQ ID NO: 15) |
| | GGCGGCTCTGAAAATGTGCTCACCCAGTCT- 3′ | |
| Reverse primer: | 5′-AGTCAGGCTAGCTTAGCTGTCCATGTGCTGGCGTTCG | (SEQ ID NO: 16) |
| | AATTTAGCAGCAGCGGTTTCTTTCGCTTTCAGCTCCAGCTT- 3′ | |

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu. Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Antibody vaccines are described in Dillman R. O. (2001) Cancer Invest. 19(8):833-841. Durrant L. G. et al. (2001) Int J. Cancer 1;92(3):414-20 and Bhattacharya-Chatterjee M, (2001) Curr. Opin. Mol. Ther. Feb;3(1):63-9 describe anti-idiotype antibodies. Many of the procedures useful for practicing the present invention, whether or not described herein in detail, are well known to those skilled in the arts of molecular biology, biochemistry, immunology, and medicine.

Monoclonal, polyclonal antibodies, peptide-specific antibodies or single chain recombinant antibodies and antigen binding fragments of any of the foregoing, specifically reacting with the tNOX isoform proteins described herein, may be made by methods known in the art. See e.g., Harlow and Lane (1988) Antibodies: A Laboratory Manual, Cold Spring Harbor Laboratories; and Goding (1986) Monoclonal Antibodies: Principles and Practice, 2d ed., Academic Press, New York.

All references cited in the present application are incorporated by reference herein to the extent that there is no inconsistency with the present disclosure. Such references reflect the skill in the arts relevant to the present invention.

The examples provided herein are for illustrative purposes, and are not intended to limit the scope of the invention as claimed herein. Any variations in the exemplified antibodies, epitopes, purification methods, diagnostic methods, preventative methods, treatment methods, and other methods which occur to the skilled artisan are intended to fall within the scope of the present invention.

### BIBLIOGRAPHY

1. Morré, D. J. (1998) in Plasma Membrane Redox Systems and Their Role in Biological Stress and Disease (Asard, H., Bérczi, A. and Caubergs, R. J., Eds) pp.121-156, Kluwer Academic Publishers, Dordrecht, Netherlands.

2. Morré, D. J. and Morré, D. M. (2003) Free Radical Res. 37: 795-808

3. Morré, D. J., Chueh, P.-J. and Morré, D. M. (1995) Proc. Natl. Acad.

Sci. USA 92:1831-1835.

4. Bruno, M., Brightman, A. O., Lawrence, J., Werderitsh, D., Morré, D. M. and Morré, D. J. (1992) Biochem. J. 284: 625-628.

5. Cho, N., Chueh, P.-J., Kim, C., Caldwell, S., Morré, D. M. and Morré, D. J. (2002) Cancer Immunol. Immunother. 51: 121-129.

6. Morré, D. J., Caldwell, S., Mayorga, A., Wu, L-Y. and Morré, D. M. (1997) Arch. Biochem. Biophys. 342: 224-230.

7. Morré, D. J. and Reust, T. (1997) J. Bioenerg. Biomemb. 29, 281-289.

8. Morré, D. J. (1995) Biochim. Biophys. Acta 1240: 201-208.

9. del Castillo-Olivares, A., Chueh, P.-J., Wang, S., Sweeting, M., Yantiri, F., Sedlak, D., Morré, D. J. and Morré, D. M. (1998) Arch. Biochem. Biophys. 358: 125-140.

10. Morré, D. J., Sedlak, D., Tang, X., Chueh, P.J., Geng, T. and Morré, D. M. (2001) Arch. Biochem. Biophys. 392: 251-256.

11. Chueh, P. J., Kim, C., Cho, N., Morré, D. M. and Morré, D. J. (2002) Biochemistry 41: 3732-3741.

12. Bird, C. (1999) Direct submission of human DNA sequence from clone 875H3 (part of APK1 antigen) to GenBank database at NCBI. (Accession no. AL049733).

13. Braman, J., Papworth, C. and Greener, A. (1996) Methods Mol. Biol. 57: 31-44.

14. Wilkinson, F. E., Kim, C., Cho, N., Chueh, P.J., Leslie, S., Moya-Camerena, S., Wu, L.-Y., Morré, D. M. and Morré, D. J. (1996) Arch. Biochem. Biophys. 336: 275-282.

15. Morré, D. J., Wu, L.-Y. and Morré, D. M. (1995) Biochim. Biophys. Acta 1240: 11-17.

16. Morré, D. J., Kim, C., Paulik, M., Morré, D. M. and Faulk, W. P. (1997) J. Bioenerg. Biomembr 29: 269-280

17. Morré, D. J., Bridge, A., Wu, L.-Y. and Morré, D. M. (2000) Biochem. Pharmacol. 60: 937-946.

18. Kozak M. (1978) Cell. 15: 1109-23.

19. Kozak, M. (1989) J. Cell Biol. 108: 229-241.

20. Tatyana, V., Borukhov, S. I. and Hellen C. (1998) Nature 394: 854-859.

21. Van der Velden, A. W. and Thomas, A. A. (1999) Int. J. Biochem. Cell Biol. 31: 87-106.

22. Suzuki, Y., Ishihara, D., Sasaki, M. Nakagawa, H., Hata, H., Tsunoda, T., Wantanabe, M., Komatsu, T., Ota, T., Isogai. T., Suyama, A., and Sugano, S. (2000) Genomics 64: 286-297.

23. Kozak, M. (1987) Nucleic Acids Res. 15: 8125-8148.

What is claimed is:

1. A method for detecting the ECTO-NOX cancer-specific tNOX isoform proteins in a biological sample, said method comprising the steps of:
(a) concentrating ECTO-NOX cancer-specific tNOX isoform proteins from a biological sample;
(b) resolving the concentrated ECTO-NOX cancer-specific tNOX proteins by isoelectric point;
(c) resolving by size the ECTO-NOX cancer-specific tNOX proteins resolved by isoelectric point; and
(d) detecting the resolved ECTO-NOX cancer-specific tNOX isoform proteins using an antibody which specifically binds cell surface NADH oxidase.

2. The method of claim 1, wherein the step of concentration is by binding to nickel agarose.

3. The method of claim 1, wherein the step of concentrating the tNOX proteins from serum or plasma is by ethanol precipitation or by ammonium sulfate precipitation.

4. The method of claim 1, wherein the concentrated ECTO-NOX cancer-specific tNOX proteins are contacted with a reducing agent.

5. The method of claim 1, wherein the step of separating the concentrated proteins is by isoelectric focusing.

6. The method of claim 1, wherein the step of size separation is by sodium dodecyl sulfate-polyacrylamide gel electrophoresis.

7. The method of claim 1, wherein the antibody is monoclonal antibody 12.1, produced by the hybridoma on deposit with the American Type Culture Collection as Accession No. PTA-4206.

8. The method of claim 1, wherein the step of detecting is using a pan isoform anti-tNOX single chain variable region (ScFv) antibody and a detectable second antibody specific for said first antibody.

9. The method of claim 8, wherein the ScFv antibody has the amino acid sequence given in SEQ ID NO: 4.

10. The method of claim 1, wherein the detectable antibody is detected by enzymatic, chromagenic, chemiluminescent, radiographic, magnetic or fluorescent methods.

11. The method of claim 8, wherein the first antibody is an S-tagged recombinant antibody and said method further comprises a step of binding a detectable second anti-S specific antibody.

12. The method of claim 8, wherein said detectable second antibody is linked to alkaline phosphatase and wherein binding is detected in the presence of a chromagenic alkaline phosphatase substrate.

13. The methods of claim 1, further comprising the step of quantifying cancer-specific tNOX isoforms by scanning and a computer-driven algorithm.

14. The method of claim 10, wherein the enzymatic method is an alkaline phosphatase method.

15. The method of claim 10, wherein the enzymatic method is a horseradish peroxidase method.

16. The method of claim 1, wherein the tNOX-specific antibody contains a recognition tag.

17. The method of claim 11, wherein the recognition tag is an S tag, a His tag, a myc tag or a NUS tag and wherein a detectable second antibody specific binds to said tag.

18. The method of claim of claim 1, wherein said biological sample is cells, serum, plasma, urine, saliva or biopsy tissue from a patient suspected of having a neoplastic condition.

19. The method of claim 1, further comprising the step of detecting the presence of tNOX isoforms of 40.5, 43, 52, 64,66,68, 75, 80 and 94 kDa, whereby a particular origin of cancer is breast cancer when a 64, 66 and/or 68 kDa tNOX isoform is detected, a particular origin of cancer is non-small cell lung cancer when a 54 kDa tNOX isoform is detected, a particular origin of cancer is small cell lung cancer when tNOX isoforms of 52 and 40.5 kDa are detected, a particular original of prostate cancer when two 75 kDa tNOX isoforms of differing isoelectric points are detected, a particular origin of cancer is cervical cancer when a 94 kDa tNOX isoform is detected, a particular origin of cancer is colon cancer when a 43 and 52 kDa tNOX isoforms are detected and a particular origin of cancer is ovarian cancer when 40.5, 52 and ca 80 kDa isoforms are detected.
